(19) United States
(12) Patent Application Publication
(10) Pub. No.: US 2010/0297673 A1
(43) Pub. Date: Nov. 25, 2010

(54) METHODS AND COMPOSITIONS FOR UPREGULATION OF PEROXIREDOXIN ACTIVITY

(75) Inventors: Ish Khanna, Alpharetta, GA (US); Ram Pillarisetti, Norcross, GA (US); Uday Saxena, Atlanta, GA (US)

Correspondence Address: Nelson Mullins Riley & Scarborough LLP;IP Department, 100 North Tryon Street, 42nd Floor, Charlotte, NC, 28202-4000, US

(73) Assignee: Reddy US Therapeutics, Norcross, GA (US)

(21) Appl. No.: 12/441,924

(22) PCT Filed: Sep. 20, 2007
(86) PCT No.: PCT/US2007/079068

§ 371 Date: Mar. 9, 2010

Related U.S. Application Data
(60) Provisional application No. 60/846,057, filed on Sep. 20, 2006.
(60) Provisional application No. 60/951,801, filed on Jul. 25, 2007.

(51) Int. Cl. G01N 33/53

(52) U.S. Cl. 435/7.21

(57) ABSTRACT

A novel method of identifying compounds capable of upregulating Peroxiredoxin activity is disclosed. The method includes providing a sample of cells that express Peroxiredoxin, providing a sample of a candidate compound, contacting the cell sample and the compound sample, and measuring a quantitative indicator of Peroxiredoxin activity within the cell sample after the contacting step. Peroxiredoxin inducers identified by the method and uses therefore to upregulate Peroxiredoxin activity in subjects and to reduce LDL and/or VLDL levels and to prevent or treat atherosclerosis and inflammatory disorders such as arthritis in subjects are also described. The invention also provides a method of treatment of inflammatory and cardiovascular diseases which comprises providing a patient in need of treatment with an effective amount of a composition that increases Peroxiredoxin protein or activity.

### CROSS REFERENCE TO RELATED PATENT APPLICATIONS

The present application claims the benefit of U.S. Provisional Application No. 60/846,057, filed Sep. 20, 2006 and U.S. Provisional Application No. 60/951,801, filed Jul. 25, 2007, each of which is relied on herein and incorporated herein by reference in its entirety.

### BACKGROUND OF THE INVENTION

(1) Field of the Invention

The present invention generally relates to pharmacological upregulation of the enzyme, Peroxiredoxin, for the treatment of conditions associated with an increase in inflammatory cytokines, including, but not limited to, inflammation-induced diseases such as arthritis, type I and type II diabetic induced vasculopathy, and asthma, and as well, can be effective for reducing low density lipoproteins (“LDL”) and very low density lipoproteins (“VLDL”) cholesterol and thereby preventing and/or treating atherosclerosis and cardiovascular disease.

(2) Description of the Related Art

As a reaction to internal physiological conditions and a variety of extracellular stimuli, including peptide growth factors and cytokines, living organisms produce reactive oxygen species (“ROS”) such as hydrogen peroxide (“H₂O₂”) and superoxide [O2.-] thereby inducing an increase in the intracellular concentration of such ROS.

While ROS serves valuable purposes such as the regulation of gene expression and cell growth and proliferation, excessive oxidative stress causes cell injury and ultimately cell death. Increased production of ROS has been implicated in the pathogenesis of inflammatory and cardiovascular diseases such as atherosclerosis, hypertension, and diabetic vascular disease. Hydrogen peroxide has also been linked to inflammatory responses and oxidant-induced stress.

In mammalian cells, potential enzymatic sources of ROS include the mitochondrial electron transport chain, the lipoxygenase and cycloxygenase enzymes, the cytochrome P450s, xanthine oxidase and NAD(P)H oxidases. O2.- generated by these systems is dismutated to H₂O₂ spontaneously or catalyzed by superoxide dismutase (SOD). Some enzymes, such as xanthine oxidase and glucose oxidase, can directly produce H₂O₂ by donating two electrons to oxygen.

H₂O₂ has several effects on vascular cells H₂O₂ is removed by enzymes, such as catalase, glutathione peroxidase, and Peroxiredoxin.

Peroxiredoxins (PRX's) are essential thiol peroxidases that reduce hydrogen peroxide at low concentrations of substrate. See Kang, et al., Trends Mol Med. 2005 December; 11(12):571-8 and Rhee, et al., IUBMB Life. 2001 July; 52(1-2):35-41. Under certain circumstances, they also reduce peroxinitrite. They are present in all organisms but also with several variants in each species. See Noguera-Mazon, et al., Photosynth Res. 2006 September; 89(2-3):277-90. The PRX family includes six isoforms in mammals. See Kang, et al., Trends Mol Med. 2005 December; 11(12):571-8. PRX's can be classified according to their enzymatic mechanism and the cysteine set involved in their catalytic cycle i.e., “1-Cys”, “typical” and “atypical 2-Cys” categories. Peroxiredoxins generally have a molecular size of 20-30 kDa.

Activation of Peroxiredoxin has been shown to reduce peroxide levels. Therefore, Peroxiredoxin plays a critical role in the regulation of peroxide-mediated signaling cascades. However, the role of Peroxiredoxin on expression of inflammatory genes has not been previously reported.

Although Peroxiredoxin's role in peroxide regulation is known, its role in the liver, especially in relation to lipid metabolism, is presently unknown. Indeed, it is not known if this enzyme affects signaling pathways associated with cholesterol metabolism.

Cholesterol is a lipid found in the cell membranes of all body tissues, and is transported in the blood plasma of all animals. Cholesterol is transported in the blood by lipoproteins and gives LDL-C, referred to as bad cholesterol and high density lipoproteins (“HDL”), referred to as good cholesterol. Cholesterol is required to build and maintain cell membranes. Cholesterol also aids in the manufacture of fat soluble vitamins, including vitamins A, D, E, and K. It is the major precursor for the synthesis of Vitamin D and of the various steroid hormones.

Elevated levels of LDL are regarded as atherogenic, or prone to cause atherosclerosis. Atherosclerosis is a disease affecting the arterial blood vessels. It is a chronic inflammatory response in the walls of the arteries, in large part due to the deposition of lipoproteins in the form of plaque. It is commonly referred to as “hardening” of the arteries. Studies have shown that high levels of LDL contribute to the formation of plaque in the arteries, while high levels of HDL prevent formation of plaque or decrease previously formed plaque in the arteries.

Statins (or HMG-CoA reductase inhibitors) form a class of hypolipidemic agents, used as pharmaceutical agents to lower cholesterol levels in people with or at risk for cardiovascular disease. They lower cholesterol by inhibiting the enzyme HMG-CoA reductase, which is the rate-limiting enzyme of the mevalonate pathway of cholesterol synthesis. Inhibition of this enzyme in the liver stimulates LDL receptors, resulting in an increased clearance of LDL from the bloodstream and a decrease in blood cholesterol levels. Despite the wide spread use of statins, a significant number of patients are still under-served in controlling plasma cholesterol. This may in part due to non-responsiveness to statins or side-effects, which may include myopathy, liver enzyme elevation and rhabdomyolysis.

Ezetimibe is another anti-hyperlipidemic medication used to lower cholesterol levels. It acts by decreasing cholesterol absorption in the intestine. It may be used alone when other cholesterol-lowering medications are not tolerated or together with statins when cholesterol levels are unable to be controlled on statins alone. Its efficacy is moderate and lowers cholesterol by 15-18%.

Therefore, new pharmacological agents for the treatment or prevention of inflammatory processes, such as atherosclerosis and arthritis are needed.

It would be desirable, therefore, to develop a screening method to identify compounds that increase the activity of Peroxiredoxin. Compounds that are identified by this method as being Peroxiredoxin activators would also be useful for lowering LDL and for prevention and/or treatment of cardiovascular disorders, such as atherosclerosis. Such compounds would also be useful for treating or preventing inflammatory disorders, such as arthritis.

### SUMMARY OF THE INVENTION

Briefly, therefore the present invention is directed to a novel method of identifying compounds capable of upregulating Peroxiredoxin activity. The method includes providing a sample of cells that express Peroxiredoxin, providing a candidate Peroxiredoxin activity-modulating compound, contacting the cell sample and the Peroxiredoxin activity-modulating compound sample in the presence of an assay for Peroxiredoxin activity, and measuring the change in Peroxiredoxin activity within the cells.

In another aspect, the present invention provides a method of identifying compounds that lower serum LDL and/or VLDL levels in a subject, the method comprising providing a sample of cells that express Peroxiredoxin; providing a sample of a candidate compound; contacting the cell sample and the candidate compound; measuring Peroxiredoxin activity within the cell sample after the contacting step; and selecting those candidate compounds that increases Peroxiredoxin activity as compounds that lower serum LDL and/or VLDL levels in the subject.

In another aspect, the present invention provides a method of reducing total and LDL-cholesterol in a cell of a subject, comprising increasing the amount and/or activity of Peroxiredoxin within the cell, wherein total and LDL-cholesterol levels are reduced.

In another aspect, the present invention provides a method of treating or preventing hypercholesterolemia and/or hypertriglyceredemia, comprising administering to a subject an effective amount of a compound that causes an increase in the amount and/or activity of Peroxiredoxin.

In another aspect, the present invention provides a method for diagnosing a dyslipidemia condition in a subject by measuring the activity of Peroxiredoxin and correlating the activity with a known dyslipidemia condition.

In another aspect, the present invention provides a novel approach to the treatment of inflammatory and cardiovascular disorders via Peroxiredoxin activation, as well as a novel means for the screening, identification and development of compounds useful in the treatment of inflammatory and cardiovascular disorders.

In a first aspect, the present invention provides a method of treating a disorder associated with an increase in inflammatory cytokines, which method comprises increasing the activity the Peroxiredoxin protein.

In another aspect, the present invention provides a method of treating a disorder associated with an increase in inflammatory cytokines, which method comprises up regulation of the Peroxiredoxin gene.

In another aspect, the present invention provides a method of treating a disorder associated with an increase in inflammatory cytokines, wherein the inflammatory cytokines is TNFα, MCP-1 or VCAM-1.

In another aspect, the present invention provides a method of treating a disorder associated with an increase in inflammatory cytokines, wherein the inflammatory cytokines is TNFα, or VCAM-1.

In another aspect the present invention provides a method of treating a disorder associated with an increase in inflammatory cytokines, wherein the disorder is an inflammatory disorder.

In a further aspect, the present invention provides a method of treating a disorder associated with an increase in inflammatory cytokines, wherein the disorder is a cardiovascular disorder.

In another aspect, the present invention provides a method of treating a disorder associated with an increase in inflammatory cytokines, wherein the disorder is a metabolic disorder.

In a further aspect, the present invention provides a method of treating a disorder associated with an increase in inflammatory cytokines, wherein the disorder is diabetic nephropathy.

In another aspect, the present invention provides a means for the screening of compounds that modulate the activity of Peroxiredoxin.

In yet another aspect, the present invention provides a method of identifying whether or not a compound is capable of increasing the activity of Peroxiredoxin.

In a further aspect, the screening and identification of compounds that provoke the activity of Peroxiredoxin, comprises (a) incubating an effective amount of the compound of interest together with Peroxiredoxin, under conditions sufficient to allow the components to interact; and (b) screening and identifying the compound by measuring the oxidation of NADPH.

In a further aspect, the screening and identification of compounds that provoke the activity of Peroxiredoxin, comprises (a) incubating an effective amount of the compound of interest together with Peroxiredoxin, NADPH, EDTA, thioredoxin, thioredoxin reductase, and Hepes-NaOH, under conditions sufficient to allow the components to interact; and (b) screening for activation of Peroxiredoxin and identifying the compound by measuring the oxidation of NADPH.

In another aspect, the method provides a means for the treatment of inflammatory-induced disease and cardiovascular disorders.

In another aspect, the present invention provides a method of treating a disease state which is alleviable by the treatment with a compound that affect the activity of Peroxiredoxin, which comprises administering to a subject in need thereof a therapeutic effective amount of a compound that increases the activity of Peroxiredoxin or a pharmaceutically acceptable salt thereof.

Yet another aspect provides a means for the treatment of inflammatory-induced disease, wherein the inflammatory-induced disease is selected from the group comprising of arthritis, asthma, atherosclerosis, irritable bowel syndrome, Crohn's disease, type 2 diabetes, psoriasis, diabetic nephropathy, retinopathy, and glomeluar nephritis.

In another aspect, the invention provides for a method of treatment of inflammatory and cardiovascular disorders which comprises providing to a patient in need of treatment an effective amount of a compound that increases the activity of Peroxiredoxin.

Alternatively, the invention provides the use of a compound that increases the activity of Peroxiredoxin for the manufacture of a medicament for the treatment of inflammatory and cardiovascular disorders.

In another aspect, the method of treatment comprises administering a compositing containing a purified amount of a compound that increases the activity of Peroxiredoxin. Such composition may be adapted to be delivered directly to the site of inflammation.

In a further aspect, the invention provides a composition comprising said compound that increases the activity of Peroxiredoxin, which composition is adapted for administration to a subject in need thereof. Such composition may be adapted to be delivered directly to the site of inflammation.

These and other aspects of the invention will be understood and become apparent upon review of the specification by those having ordinary skill in the art.

### BRIEF DESCRIPTION OF THE DRAWINGS

FIG. 1 illustrates the results from Example 1 in which a Peroxiredoxin activity knock-down experiment decreased LDL clearance by liver cells.

FIG. 2 illustrates the results from Example 2 in which a Peroxiredoxin activity knock-down experiment in animals increased plasma LDL and apoB concentrations.

FIG. 3 illustrates the results from Example 3 in which Compound A increased Peroxiredoxin activity in liver cells.

FIG. 4 illustrates the results from Example 4 in which compound A decreased total cholesterol, LDL and triglycerides in LDL-receptor null mice models of hypercholesterolemia.

FIG. 5 also illustrates the results from Example 4 in which Compound A decreased triglycerides in apolipoprotein E-null mice models of hypercholesterolemia.

FIG. 6 illustrates an image of peroxiredoxin activity.

FIG. 7 illustrates the role of Stat1 overexpression on LDL uptake.

FIG. 8 illustrates the role of Stat1 overexpression Perlecan levels and LDLr levels.

FIGS. 9-12 illustrate the role of H₂O₂ and Perlecan in LDL uptake by liver cells.

FIG. 13 illustrate the role of Peroxiredoxin knock-down on Perlecan levels and LDLr levels.

FIGS. 14 and 15 illustrate that H₂O₂ and Peroxiredoxin I regulate Stat1 activity.

FIG. 16 illustrates that Peroxiredoxin-activating compounds decrease hydrogen peroxide levels in cells.

FIG. 17 illustrates that Peroxiredoxin-activating compounds inhibit inflammatory cytokine expression in endothelial cells.

### DETAILED DESCRIPTION OF THE PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The present invention has discovered that compounds which induce Peroxiredoxin activity reduce inflammatory cytokine expression in endothelial cells and macrophages. This has led to the discovery that Peroxiredoxin plays role in modulating inflammatory cytokine expression. Also, for the first time, the present invention has discovered that Peroxiredoxin/hydrogen peroxide signaling has an impact on cholesterol clearance by liver cells. Thus, Peroxiredoxin plays an important role in facilitating LDL clearance by liver cells and thereby reducing plasma cholesterol levels.

Inflammation is the underlying cause of many vascular diseases including atherosclerosis and diabetic vascular disease. An increase of molecules involved in endothelial inflammation, such as TNFα, VCAM-1 and monocyte chemoattractant protein-1 (MCP-1) leads to endothelial dysfunction and angiogenesis. An overall approach to the understanding and treatment of these diseases and its complications will be to block the increase of these molecules. VCAM-1 is a pro-inflammatory cytokine that is known to play a key role in the pathogenesis of atherosclerosis and other inflammatory diseases including arthritis and asthma MCP-1, another pro-inflammatory cytokine is found highly expressed in human atherosclerotic lesions and postulated to play a central role in monocyte recruitment into the arterial wall and developing lesions. Recent results suggest that MCP-1 is also a key pathogenic molecule in diabetic nephropathy. The levels of urinary MCP-1 in patients with the advanced stage were significantly higher than those in patients with the mild stage of the disease, or in healthy controls.

The present invention relates to a method for treating disease states caused the excess expression of inflammatory cytokines, such as MCP-1 and VCAM-1. More specifically, the present invention relates to a method for preventing and/or reducing cellular and tissue damage caused when inflammatory cytokines are released in response to various disease states or pathologies. The method of the present invention is useful in preventing and treating a variety of disease states or pathological situations in which inflammatory cytokines are produced and released. The method of the present invention contemplates reducing inflammatory damage by activating Peroxiredoxin.

It is one of the surprising discoveries of the present invention that compounds that activate Peroxiredoxin, within a subject, can facilitate the treatment and recovery of individuals suffering from a variety of medical conditions. The conditions contemplated as treatable under the present invention result from a disparate number of etiological causes. Nevertheless, they share a common feature in that their pathological conditions are either caused or exacerbated by inflammatory cytokines. Thus, the administration of compounds that activate Peroxiredoxin provide an effective treatment for a variety of medical conditions.

Such conditions include but are not limited to: cardiovascular disorders and inflammatory disorders where ROS are believed to play a detrimental role such as arthritis, asthma, atherosclerosis, irritable bowel syndrome, Crohn's disease, type 2 diabetes, psoriasis, diabetic nephropathy, retinopathy, and glomeluar nephritis.

Compounds that modulate the activity of Peroxiredoxin may be identified by an effective amount of the compound of interest together with NADPH, EDTA, thioredoxin, thioredoxin reductase Hepes-NAOH and Peroxiredoxin, under conditions sufficient to allow the components to interact; and (b) screening and identifying the compound by measuring the oxidation of NADPH. Peroxiredoxin reduces the amount of H₂O₂ and such activity is coupled to the oxidation of NADPH. Oxidation of NADPH may be measured as a decrease in absorbance at 340 nm. Compounds that modulate the activity of Peroxiredoxin are identified and selected. Such compounds can be formulated for administration to a patient in need of treatment.

Thus, another embodiment provides for a method of treating a disease state which is alleviable by the treatment with a compound that is identified as affecting the activity of the Peroxiredoxin protein or gene, which comprises administering to a subject in need thereof a therapeutic effective amount said compound or a pharmaceutically acceptable salt thereof. Such disease state includes, but is not limited to metabolic disorders, cardiovascular disorders and inflammatory-induce disease, including but not limited to arthritis, asthma, atherosclerosis, irritable bowel syndrome, Crohn's disease, type 2 diabetes, psoriasis, diabetic nephropathy, retinopathy, and glomerular nephritis.

In another aspect, the present invention is directed to a method of identifying compounds capable of upregulating Peroxiredoxin activity. The method includes proving purified Peroxiredoxin or a sample of cells that express Peroxiredoxin, providing a sample of a Peroxiredoxin activity-modulating candidate compound (a “candidate compound”), contacting Peroxiredoxin and the compound sample in the presence of an assay for Peroxiredoxin activity, and measuring the change in Peroxiredoxin activity that is caused by the contact with the candidate compound. It has been found to be useful to use a high-throughput assay based on hydrogen peroxide conversion of NADPH to NADP through thioredoxin system as the measure of a quantitative indicator (NADPH) of the change in Peroxiredoxin activity within the cell sample that is caused by the candidate compound. Absorbance spectroscopy can be used to monitor the change in concentration of the quantitative indicator, NADPH, as it is converted to NADP in the presence of hydrogen peroxide. For example, a Peroxiredoxin inducer will cause the concentration of NADPH to be reduced over time in the aforementioned assay.

The Peroxiredoxin family of enzymes is known to include six isoforms in mammals, Peroxiredoxin I-VI. As used herein, the term Peroxiredoxin shall be interpreted as including one or more of the Peroxiredoxin family of enzymes unless explicitly stated otherwise.

In a preferred embodiment, the Peroxiredoxin enzyme is the Peroxiredoxin I enzyme. In a preferred embodiment, the present invention is a method of identifying compounds capable of upregulating Peroxiredoxin activity. In a preferred embodiment, the present invention is a method of identifying compounds capable of upregulating Peroxiredoxin I activity. In one embodiment, the cells that express Peroxiredoxin are human liver cells. In one embodiment, the quantitative indicator of Peroxiredoxin activity is luciferase activity. In a preferred embodiment, the quantitative indicator of Peroxiredoxin activity is NADPH levels.

In another embodiment, the step of contacting the cell sample and the candidate sample in the presence of a high-throughput assay based on luciferase activity includes contacting the cell sample and the candidate sample in which the luciferase gene is joined to a Peroxiredoxin promoter in an expression vector that is transfected into cells. When the sample candidate successfully upregulates the Peroxiredoxin activity, expression of the luciferase reporter is increased and measured through an enzymatic release of light. In these embodiments, the quantitative activity that is measured is the light given off by the expressed luciferase.

The quantitative indicator may be direct measurement of Peroxiredoxin activity or levels of Peroxiredoxin protein in the cells.

In the present screening method, an increase in the monitored quantitative indicator indicates an upregulation of Peroxiredoxin activity. As used herein, the terms “Peroxiredoxin activity” refer to the amount of or concentration of Peroxiredoxin enzyme and/or the activity of the Peroxiredoxin enzyme in the reduction of intra/extracellular hydrogen peroxide levels. Accordingly, in the present method, when the monitored quantitative indicator indicates an increase in Peroxiredoxin activity upon contact of the cell sample with the candidate compound, the candidate compound is shown to be effective in upregulating the Peroxiredoxin activity.

In other aspects of the present invention, it has been shown that an upregulation in Peroxiredoxin activity results in a lowering of serum LDL and/or VLDL levels. A reduction in LDL and/or VLDL serum levels may serve to prevent and/or reduce plaque build-up in arteries and prevent and/or reduce complications resulting from having excessive plaque build-up in arteries. In one aspect, therefore, the invention is directed to a method of preventing and/or reducing plaque build-up in arteries by administering to a subject a Peroxiredoxin activity inducer.

In another aspect, the invention provides a method of lowering serum LDL and/or VLDL levels by administering to a subject a Peroxiredoxin inducer. As used herein, the term “Peroxiredoxin inducer” will be understood by those having ordinary skill in the art as including any compound that increases Peroxiredoxin activity. By way of example, any compound that causes an increase in Peroxiredoxin activity identified by the present method of identifying Peroxiredoxin inducers that is described herein is considered to be a Peroxiredoxin inducer.

The term “treatment” or “treating” as used herein covers any treatment of a disease in a mammal, particularly a human, and includes: (i) preventing the disease from occurring in a subject which may be predisposed to the disease, but has not yet been diagnosed as having it; (ii) inhibiting the disease, i.e., arresting its development; or (iii) relieving the disease, i.e., causing regression of the disease.

One illustrative Peroxiredoxin inducer is Compound A, which has the structure:

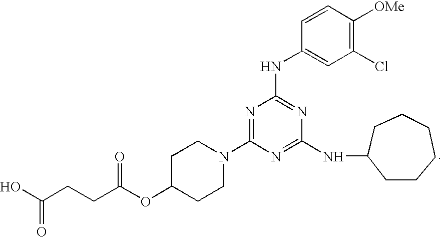

In yet another aspect, the invention provides a method of reducing total cholesterol and LDL-cholesterol in a cell of a subject, which method comprises increasing the level of Peroxiredoxin within the cell thereby reducing serum cholesterol levels.

In another aspect, the invention provides a method of treating or mitigating hypercholesterolemia and hyper-triglyceredemia, the method including administering to a subject having the disorder an effective amount of a compound that causes an increase in the level of Peroxiredoxin.

In another aspect, the invention provides a method of treating or mitigating hypercholesterolemia and hyper-triglyceredemia, the method including administering to a subject having the disorder an effective amount of a compound that causes an increase in the activity of Peroxiredoxin.

In yet another aspect, the invention provides a method of identifying compounds capable of increasing the activity of Peroxiredoxin, wherein a candidate compound is contacted with Peroxiredoxin and the Peroxiredoxin activity is measured by determining the levels of NADP that are formed.

The present application provides compounds, compositions and methods that increases Peroxiredoxin activity and decreases circulating LDL levels. In one aspect, the present invention encompasses compounds which decrease total cholesterol, LDL and/or triglycerides in subjects suffering from hypercholesterolemia. In another aspect, the present invention encompasses methods and kits for diagnosing a dyslipidemia condition in a subject by measuring the activity of Peroxiredoxin and correlating the activity with a known dyslipidemia condition.

Despite a strong association between inflammation and hyperlipidemia, the cause and effect of this relationship is not known. Hydrogen peroxide (“H₂O₂”) is an important second messenger in signal transduction pathway of many inflammatory cytokines. The present invention suggests that H₂O₂ signaling impacts LDL uptake by the liver. Exposure of liver cells to non-toxic levels of hydrogen peroxide led to decreased LDL uptake. H₂O₂ did not change the expression of LDL receptor but decreased the expression of perlecan, a heparin sulfate proteoglycan accessory lipoprotein receptor present in liver sinusoids. Peroxiredoxin 1 plays a critical role in the regulation of H₂O₂-signaling. Consistent with this Peroxiredoxin 1 knock down in liver cells was associated diminished uptake of LDL and decreased perlecan expression. Knock down of catalase, another H₂O₂ degrading enzyme, in contrast had no effect on perlecan expression. Elevated H₂O₂ levels activated STAT1, a known transcriptional suppressor of Perlecan indicating a mechanism of regulation of Perlecan expression by H₂O₂. In vivo, liver specific knock down of Peroxiredoxin resulted in a significant increase in plasma LDL cholesterol and apoB protein levels without changes in apoB mRNA. These data suggest that H₂O₂-Peroxiredoxin-Stat1-perlecan pathway regulates plasma LDL/apoB. The present invention suggests that during inflammation dysregulation of H₂O₂ based signaling cascade leads to hyperlipidemia.

Elevated plasma cholesterol levels are a major contributing factor to atherosclerotic cardiovascular disease. Statins which inhibit cholesterol biosynthesis promote hepatic clearance of plasma LDL-c through LDL-receptor-mediated processes and this enhanced clearance is a major contributing factor to lowering of plasma cholesterol. Both genetic and dietary factors contribute to elevation of blood cholesterol. In addition, systemic inflammation is often associated with hyperlipidemia, although the exact mechanism behind this association is not clear. In metabolic syndrome, subclinical inflammation is often present and is correlated with hyperlipidemia. In addition dietary fat has direct effects on inflammatory markers in humans. Although cytokines differ in their mode of action, recent data suggest that many generate hydrogen peroxide in their signaling cascade. Hydrogen peroxide is considered an effective signaling molecule because it is rapidly produced and is easily controlled by antioxidant enzymes. It is also very reactive and its reactivity with thiol groups on proteins in part contributes to H₂O₂ regulation of transcription factor activity. Transient elevation of H₂O₂ is thought to inactivate phosphatases leading to sustained presence of active phosphorylated forms of transcription factors.

Although the role of H₂O₂ is well studied in vascular dysfunction, its role in liver especially in relation to lipid metabolism is not known. The present invention shows that H₂O₂ reduces LDL uptake by liver cells. It is also shown that Peroxiredoxin 1, an intercellular enzyme that dissipates H₂O₂, is critical for eliminating H₂O₂ and restoring liver's capacity to clear apoB-lipoproteins in vitro and in vivo.

One objective of the study was to identify a molecular link between inflammation and dyslipidemia. Research in the past few years has convincingly demonstrated a critical role for H₂O₂ in the signaling cascade of many growth factors and cytokines. Because H₂O₂ is an important component of inflammation, its role in LDL uptake by liver cells was explored. The data demonstrated that H₂O₂ has a negative impact on LDL uptake. Addition of non-toxic amounts of H₂O₂ significantly blunted LDL uptake in the liver cells. Although LDLr is the major receptor for LDL in liver cells, H₂O₂ did not affect LDLr expression but significantly decreased perlecan expression, the most copious liver HSPG. Liver HSPGs have been postulated to play a role in triglyceride and remnant lipoprotein clearance. This is further confirmed in the recent studies by MacArthur, et al., showing that HSPGs under normal physiological conditions are critically important in the clearance of VLDL and remnant lipoproteins, independent of LDLR family members. The principal proteoglycan in Disse's space, the site of hepatic lipoprotein trapping, appears to be perlecan. Both hepatocytes and endothelial cells may contribute to its synthesis in liver. Immunoelectron microscopy revealed perlecan at the basement membranes surrounding bile ducts and blood vessels, and in the space of Disse discontinuously interacting with hepatocyte microvilli. In diabetic animals, a decrease of liver HSPG was attributed to diabetic dyslipidemia and perlecan was postulated to be a candidate HSPG. The present invention demonstrated that perlecan is important for LDL uptake in liver cells.

The role of endogenous H₂O₂ signaling in liver cell LDL uptake was further confirmed by Peroxiredoxin knock down experiments. RNAi-mediated reduction in Peroxiredoxin expression but not catalase expression resulted in decreased perlecan mRNA and LDL uptake. Whereas catalase is mostly confined to the peroxisome, Peroxiredoxins are abundant in the cytosol. During catalysis of H₂O₂ reduction, the active-site residue, Cys-SH, of Peroxiredoxin reacts with two molecules of H₂O₂, and thus becomes hyperoxidized to Cys-SOOH. Consequently, Peroxiredoxins are inactivated. This inactivation, which can be reversed by sulfuredoxin, may represent a built-in mechanism to prevent damping of the H₂O₂ signal. Thus, Peroxiredoxin-1's location within the vicinity of receptors is ideally positioned to regulate locally generated H₂O₂ and thereby modulate LDL uptake in liver.

In vitro observations on the role of Peroxiredoxin were further confirmed in vivo using RNAi approach. Intravenous injection of RNAi predominantly localizes in liver and consistent with this, a robust 60% reduction was seen in Peroxiredoxin mRNA in mice liver injected with Peroxiredoxin-RNAi. A significant decrease in liver perlecan mRNA was also noted in these animals confirming the relationship of Peroxiredoxin with perlecan. Peroxiredoxin-KO mice showed elevated plasma cholesterol and apoB levels. Coupled with the in vitro observations in liver cells, these data suggest that the increased plasma apoB is due decreased clearance of lipoproteins in Peroxiredoxin knock-down mice.

The activity of many enzymes and transcription factors is governed by the phosphorylation state and protein tyrosine phosphatases (PTPs) play an important role in the regulation of protein phosphorylation. The PTP family features a common Cys-X-X-X-X-X-Arg active-site motif. The conserved catalytic cysteine possesses a low pKa and exists as a thiolate anion with enhanced susceptibility to oxidation by H₂O₂. Oxidation of the essential cysteine abolishes phosphatase activity. Reversible inactivation of different PTPs has been demonstrated in cells stimulated with growth factors and cytokines. Oxidative inactivation of these phosphatases and increased tyrosine phosphorylation of target proteins were found to be dependent on H₂O₂ production. Stat1 it is not only sensitive to H₂O₂ signaling, but is also known to be a transcriptional repressor of perlecan gene expression. Stat1 is activated by phosphorylation and inactivated via dephosphorylation by PTP. Elevated H₂O₂ (exogenous or by Peroxiredoxin knock-down) induced Stat1 promoter activity in liver cells. More importantly overexpression of Stat1 not only reduced perlecan expression, but significantly reduced liver cell LDL uptake. Although it is conceivable that inactivation of PTPs would activate other transcription factors or signaling kinases, the magnitude of effect seen on perlecan expression and LDL uptake in Stat1 overexpressing cells strongly suggest that Stat1 is the major mediator of H₂O₂ effects on LDL uptake.

The present invention provides new information on how lipid metabolism may be dysfunctional during inflammation. Lymphotoxin (LT) and LIGHT are regulators of key enzymes that control lipid metabolism. Dysregulation of LIGHT expression on T cells resulted in hypertriglyceridemia and hypercholesterolemia and inhibition of LT signaling attenuate dyslipidemia. The present invention indicates the downstream effect of such inflammatory mediators. Inflammatory stimulus triggered H₂O₂ generation during inflammation could overpower the Peroxiredoxin 1 H₂O₂ scavenging system and reduce the uptake of LDL. Conversely over expression or activation of Peroxiredoxin 1 could attenuate the H₂O₂ signaling cascade and enhance LDL uptake.

The present invention also opens up new avenues for LDL lowering. Despite the wide use of statins, achieving ATP III recommended guidelines for cholesterol management continues to be a daunting task. This may in part be due to mechanism related (resistant to statins) or physicians unwillingness to use high dose of statins due to safety concerns. Thus, newer mechanisms that complement existing cholesterol lowering drugs would be of great benefit. Studies presented here identify novel pathways to lower cholesterol independent of HMGCoA reductase or LDL receptor. Pharmacological activation of Peroxiredoxin activity to reduce excess production of endogenous H₂O₂ levels could promote LDL receptor-independent clearance of LDL and lowering of plasma cholesterol. Similarly, inhibition of Stat1 could lower plasma cholesterol.

Therefore, in another embodiment the present invention provides a novel method of identifying compounds capable of a decreasing Stat1 activity, the method comprising providing a sample of cells that express Stat1, providing a sample of a candidate compound, contacting the cell sample and the candidate compound; and measuring Stat1 activity within the cell sample after the contacting step to identify those compounds that decrease Stat1 activity.

In another embodiment the present invention provides a method of identifying compounds that lower serum LDL and/or VLDL levels in a subject, the method comprising providing a sample of cells that express Stat1, providing a sample of a candidate compound, contacting the cell sample and the candidate compound, measuring Stat1 activity within the cell sample after the contacting step, and selecting those candidate compounds that decrease Stat1 activity as compounds that lower serum LDL and/or VLDL levels in the subject.

In another embodiment the present invention provides a method of lowering serum LDL and/or VLDL levels in a subject comprising administering to the subject a Stat1 inhibitor.

In another embodiment the present invention provides a method of lowering serum triglyceride levels in a subject comprising administering to the subject a Stat1 inhibitor.

In another embodiment the present invention provides a method of reducing total and LDL-cholesterol in a cell of a subject, comprising decreasing the amount and/or activity of Stat1 within the cell, wherein total and LDL-cholesterol levels are reduced.

In another embodiment the present invention provides a method of treating or preventing hypercholesterolemia and/or hypertriglyceredemia, comprising administering to a subject an effective amount of a compound that causes a decrease in the amount and/or activity of Stat1.

In another embodiment the present invention provides a method for diagnosing a dyslipidemia condition in a subject by measuring the activity of Stat1 and correlating the activity with a known dyslipidemia condition.

For ease of reference, the present invention will be described with reference to administration to human subjects. It will be understood, however, that such descriptions are not limited to administration to humans, but will also include administration to other animals, such as mammals, unless explicitly stated otherwise.

The present method includes administering one or more Peroxiredoxin inducers and/or Stat1 inhibitors to the subject by administration means known in the art. Administration means contemplated as useful include one or more of topically, buccally, intranasally, orally, intravenously, intramuscularly, sublingually, and subcutaneously. Other administration means known in the art are also contemplated as useful in accordance with the present invention and are discussed in more detail below.

In some embodiments, it may be useful to include one or more of the Peroxiredoxin inducers and/or Stat1 inhibitors as a salt. Those having ordinary skill in the art will recognize the salts of the Peroxiredoxin inducer compounds.

In some embodiments, the composition may be an aqueous composition. The composition may also be nebulized or aerosolized.

The subject invention involves the use of an effective amount of one or more Peroxiredoxin inducers for lowering serum LDL and/or VLDL levels, thereby treating or preventing atherosclerosis and other conditions caused by higher than normal levels of LDL and/or VLDL in subjects having higher than normal levels of LDL and/or VLDL, subjects having plaque-build-up in arteries, subjects suffering from atherosclerosis, and subjects in need of prevention of atherosclerosis.

An exemplary method of administering one or more Peroxiredoxin inducers is topical, intranasal administration, e.g., with nose drops, nasal spray, or nasal mist inhalation. Other exemplary methods of administration include one or more of topical, bronchial administration by inhalation of vapor and/or mist or powder, orally, intravenously, intramuscularly, and subcutaneously.

Other ingredients which may be incorporated in the present invention include safe and effective amounts of preservatives, e.g., benzalkonium chloride, thimerosal, phenylmercuric acetate; and acidulants, e.g., acetic acid, citric acid, lactic acid, and tartaric acid. The present invention may also include safe and effective amounts of isotonicity agents, e.g., salts, such as sodium chloride, and more preferably non-electrolyte isotonicity agents such as sorbitol, mannitol, and lower molecular weight polyethylene glycol.

In the present method, a subject in need of lowering serum LDL and/or VLDL is treated with an amount of one or more Peroxiredoxin inducers, where the amount of the one or more Peroxiredoxin inducers and/or Stat1 inhibitors provides a dosage or amount that is sufficient to constitute a treatment or prevention effective amount.

As used herein, an “effective amount” means the dose or amount of a Peroxiredoxin inducer to be administered to a subject and the frequency of administration to the subject which is readily determined by one of ordinary skill in the art, by the use of known techniques and by observing results obtained under analogous circumstances and has some therapeutic action. The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the subject to be treated, and other relevant circumstances.

The phrase “therapeutically-effective” indicates the capability of an agent to prevent, or improve the severity of, the disorder, while avoiding adverse side effects typically associated with alternative therapies.

The one or more Peroxiredoxin inducers can be supplied in the form of a novel therapeutic composition that is believed to be within the scope of the present invention.

When the one or more Peroxiredoxin inducers and/or Stat1 inhibitors are supplied along with a pharmaceutically acceptable carrier, a pharmaceutical composition is formed. A pharmaceutical composition of the present invention is directed to a composition suitable for the prevention or treatment of the disorders described herein. The pharmaceutical composition comprises at least a pharmaceutically acceptable carrier and one or more Peroxiredoxin inducers. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in present invention are the isomeric forms and tautomers and the pharmaceutically-acceptable salts of Peroxiredoxin inducers. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, p-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group IA) salts, alkaline earth metal (Group IIA) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of any of the diseases or disorders described above. Besides being useful for human treatment, these combinations are also useful for treatment of mammals, including horses, dogs, cats, rats, mice, sheep, pigs, etc.

The term “subject” for purposes of this application includes any animal. The animal is typically a human. A preferred subject is one in need of treatment or prevention of the disorders discussed herein.

For methods of prevention, the subject is any human or animal subject, and preferably is a subject that is in need of prevention and/or treatment of atherosclerosis or other disorders caused by high levels of LDL and/or VLDL. The subject may be a human subject who is at risk of disorders such as those described above. The subject may be at risk due to genetic predisposition, sedentary lifestyle, diet, exposure to disorder-causing agents, exposure to pathogenic agents and the like.

The present pharmaceutical compositions may be administered enterally and/or parenterally. Parenteral administration includes subcutaneous, intramuscular, intradermal, intramammary, intravenous, and other administrative methods known in the art. Enteral administration includes solution, tablets, sustained release capsules, enteric coated capsules, syrups, beverages, foods, and other nutritional supplements. When administered, the present pharmaceutical composition may be at or near body temperature.

The phrase “therapeutically-effective” and “effective for the treatment, prevention, or inhibition,” are intended to qualify the amount of each agent for use in the therapy which will achieve the goal of increased proteoglycan levels, while avoiding adverse side effects typically associated with alternative therapies.

In particular, the Peroxiredoxin inducers and/or Stat1 inhibitors of the present invention, or compositions in which they are included, can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, any of a variety of herbal extracts, milk, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing one or more Peroxiredoxin inducers and/or Stat1 inhibitors may be formulated with sweetening agents, for example glycerol, sorbitol or sucrose. Such formulations may also contain a demulcent, a preservative, and flavoring and coloring agents.

The subject Peroxiredoxin inducers and/or Stat1 inhibitors and compositions in which they are included can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing or wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables;

The subject Peroxiredoxin inducers and/or Stat1 inhibitors and compositions in which they are included can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The subject Peroxiredoxin inducers and/or Stat1 inhibitors and compositions in which they are included can also be administered topically, in the form of creams, ointments, jellies, collyriums, solutions, patches, or suspensions.

Daily dosages of the Peroxiredoxin inducers and/or Stat1 inhibitors can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems in addition to nutritional supplements include sprays, capsules, tablets, drops, and gelatin capsules, for example.

Those skilled in the art will appreciate that dosages for the therapeutic use of the Peroxiredoxin inducers may also be determined with guidance from Goodman & Goldman's The Pharmacological Basis of Therapeutics, Ninth Edition (1996), Appendix II, pp. 1707-1711.

Preferred dosages for the Peroxiredoxin inducers and/or Stat1 inhibitors are those that are effective to lower serum LDL and/or VLDL levels. In especially preferred embodiments, the dosage should be in a concentration effective to induce the activity of Peroxiredoxin and/or decrease the activity of Stat1 such that plaque build-up in the arteries is reduced. In yet another embodiment an effective dosage is an amount that is effective to lower serum LDL and/or VLDL levels in the subject. In another embodiment, an effective dosage is an amount that is effective to upregulate Peroxiredoxin activity and/or reduce Stat1 activity in the subject.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLES

Human hepatocellular carcinoma liver cells (HepG2) and Dulbecco's Modification of Eagle's Medium (DMEM) was purchased from ATCC. FBS (Fetal Bovine Serum), Pen/strep (Penicillin-Streptomycin) human low-density lipoprotein complexed with 1,1′-dioctadecyl-3,3,3′,3′-tetramethyl indocarbocyanine perchlorate (Dil LDL), 4′,6-diamidino-2-phenylindole, dilactate (DAPI), and clearmount was purchased from Invitrogen. Delipidated calf serum (DLCS) and DMSO was purchased from Sigma. Luciferase Assay System was purchased from Promega. Luciferase Reporter Vectors was purchased from Panomics. Fugene 6 transfection reagent was purchased from Roche. RNAqueous kit, Peroxiredoxin I specific siRNA, and Silencer siRNA transfection II kit was purchased from Ambion. Full Velocity SYBR Green QRT-PCR Master Mix and primer sets was purchased from Stratagene.

Dil-LDL Uptake Assay:

Despite a strong association between inflammation and hyperlipidemia, the cause and effect of this relationship is not known. Hydrogen peroxide is an important second messenger in signal transduction pathway of many inflammatory cytokines. The present invention has shown that H₂O₂ signaling impacts LDL uptake by liver. Exposure of liver cells to non-toxic levels of hydrogen peroxide (H₂O₂) led to decreased LDL uptake. H₂O₂ did not change the expression of LDL receptor but decreased the expression of perlecan, a heparan sulfate proteoglycan accessory lipoprotein receptor present in liver sinusoids.

Peroxiredoxin 1 plays a critical role in the regulation of H₂O₂-signaling. Consistent with this, Peroxiredoxin 1 knock-down in liver cells was associated diminished uptake of LDL and decreased perlecan expression. Knock-down of catalase, another H₂O₂ degrading enzyme, in contrast had a no effect on perlecan expression. Elevated H₂O₂ levels activated STAT1, a known transcriptional suppressor of Perlecan indicating a mechanism of regulation of Perlecan expression by H₂O₂. In vivo, liver specific knock-down of Peroxiredoxin resulted in a significant increase in plasma LDL cholesterol and apoB protein levels without changes in apoB mRNA. These data suggest that H₂O₂-Peroxiredoxin-Stat1-perlecan pathway regulates plasma LDL/apoB. During inflammation dysregulation of H₂O₂ based signaling cascade leads to hyperlipidemia.

Elevated plasma cholesterol levels are a major contributing factor to atherosclerotic cardiovascular disease. Statins which inhibit cholesterol biosynthesis promote hepatic clearance of plasma LDL-c through LDL-receptor-mediated processes and this enhanced clearance is a major contributing factor to lowering of plasma cholesterol. Both genetic and dietary factors contribute to elevation of blood cholesterol. In addition, systemic inflammation is often associated with hyperlipidemia, although the exact mechanism behind this association is not clear. In metabolic syndrome, subclinical inflammation is often present and is correlated with hyperlipidemia. In addition dietary fat has direct effects on inflammatory markers in humans. Although cytokines differ in their mode of action, recent data suggest that many generate hydrogen peroxide in their signaling cascade. Hydrogen peroxide is considered an effective signaling molecule because it is rapidly produced and is easily controlled by antioxidant enzymes. It is also very reactive and its reactivity with thiol groups on proteins in part contributes to H₂O₂ regulation of transcription factor activity. Transient elevation of H₂O₂ is thought to inactivate phosphatases leading to sustained presence of active phosphorylated forms of transcription factors.

Although the role of H₂O₂ is well studied in vascular dysfunction, its role in liver especially in relation to lipid metabolism is not known. Here it has been shown that H₂O₂ reduces LDL uptake by liver cells. It has also been shown that Peroxiredoxin 1, an intercellular enzyme that dissipates H₂O₂, is critical for eliminating H₂O₂ and restoring liver's capacity to clear apoB-lipoproteins in vitro and in vivo.

Glass cover slips were autoclaved and aseptically transferred to 6-well tissue culture plates in a tissue culture hood. HepG2 cells were prepared from the T-75 flasks and were used to seed the 6-well plates (containing sterile cover slips) with 7.5×10⁵ cells per well per 1 ml of growth media. The following day, the media was removed and the wells were washed 3 times with 2 mL DMEM+10% DLCS very gently. After washes, 2 ml of DMEM+10% (DLCS) was added to the wells and treatments continued for 24 hours. In selected experiments, cells were treated with 100 uM H2O2, control IgG or perlecan antibodies. The next day, the media was not changed and Dil LDL and DAPI were added to wells for uptake (5 hours). After the uptake was completed, the wells were washed gently 3 times with PBS (2 mL/well/wash) and after last wash, cells were fixed with 1 mL of 4% formaldehyde in PBS for 20 minutes. Wells were washed 5 times with water in excess. Cover slips were then mounted to slides using clearmount. Slides were stored at −20° C. The slides were imaged using the Nikon microscope and ColdSNAP-Pro camera. All pictures were taken using 500 millisecond exposure setting using Image Pro Plus. Images were quantitated using Image Pro-Plus software. The intensity per area of the fluorescent label was quantitated using the software and was used to generate the figure which is LDL uptake per area as a percent of the control.

Stat1 Activity Assay:

HepG2 cells were prepared from the T-75 flasks and were used to seed the 24-well plates with 7.6×10⁴ cells per well per 0.5 ml of DMEM+10% FBS. The next day, Stat1-lucuferase transfections were set up with Fugene6 according to the manufacturer's directions. In brief, transfection mixtures were prepared in DMEM in separate tubes from the reporter constructs also in DMEM. The samples incubated for 5 minutes at room temperature. Then, the constructs were mixed with the Fugene6 and incubated again for 30 minutes at room temperature. Then 50 μl transfection mix was added to each well in the existing media. Cells were allowed to incubate over night. In selected experiments, cells were treated with 100 uM H2O2 as well as with siPRDX1. After treatment, cells were lysed with the Promega lysis buffer. The wells were scraped and lysates were transferred into pre-chilled tubes and the lysates were cleared via centrifugation. 20 μl of cleared lysate was added to wells in 96 well clear plates and 100 μl of luciferase assay buffer was added to wells. Relative light units were counted using the PerkinElmer Envision.

Real Time-PCR:

HepG2 cells were prepared from the T-75 flasks and were used to seed the 24-well plates with 1.6×10⁵ cells per well per 1 ml of growth media. The next day, the media was removed and 1 ml of low-serum growth media was added to each well for 24 hours. The following day, the low-serum media was removed. In selected experiments, cells were treated with 100 uM H₂O₂ or si-Peroxiredoxin I. Media was removed and cells were lysed with the lysis buffer from the Ambion RNAqueous kit and plate was placed on ice. RNA was extracted using the kit according to the protocol. The RNA was quantitated using a spectrophotometer and frozen at −80° C. The next day, the RNA was used for real-time PCR. The RNA was thawed and master mixes for each primer were prepared with the Stratagene Full Velocity SYBR Green QRT-PCR Master Mix. Master Mix with primer was added to the appropriate wells then RNA was added to appropriate wells. The reaction plate was inserted into the MX3000p instrument running the SYBR green program of the MXPro qper software (Stratagene). The CT values generated from the real time PCR using the MXPro qper software were used to generate fold increase by the ΔΔCT method.

siRNA Mediated Silencing of Peroxiredoxin I:

HepG2 cells were grown in T-75 flasks prior to study. Transfection of Peroxiredoxin I specific siRNA in 24-well plates was performed according to the manufacturer's instruction. After 48 hrs, RNA was isolated and RT-PCR was performed to examine changes in message levels all normalized to actin. In other experiments, three daily consecutive treatments of siRNA was administered in C57BL/6 mice by tail vein injection according to the manufacturer's protocol. Fasted blood samples were collected from each animal at baseline, and at the end of the study. Total cholesterol and apoB levels were measured using an enzymatic assay. Percentage change was evaluated by comparing to both the baseline and the vehicle at the end of the study. In addition, liver tissue was collected at the end of the study. RNA was isolated according to the manufacturer's protocol (Qiagen) and analyzed for changes in gene expression normalized to actin using RT-PCR.

Adenoviral Overexpression of Stat1:

Cultured HepG2 cells were infected with either a Stat1 (Vector Biolabs) or null virus at a multiplicity of infection of 200 according to the manufacturer's protocol. LDL uptake and RT-PCR was then performed as stated above.

Statistical Analyses:

In this study, numerous independent experiments were performed, with similar results, and one representative experiment is shown in each of the figures. The quantitative LDL uptake data, ELISA data, and the relative mRNA expression levels detected by real-time RT-PCR are expressed as the mean±SD. Statistical significance was determined by the paired Student's t-test. Differences were considered to be statistically significant for p<0.05.

Role of H₂O₂ in Uptake of LDL by Liver Cells:

In cells H₂O₂ is generated in response to a variety of stimuli including cytokines and growth factors. When liver cells were exposed to low concentration of H₂O₂ that does not cause any toxicity, the cell ability to take up LDL particles was significantly decreased. Lipoprotein uptake in liver cells is mediated by the high affinity LDL-receptor and/or low affinity/high capacity HSPG receptors present in the liver space of disse. H₂O₂ treatment did not have a significant effect on LDLr mRNA levels, but significantly reduced the expression levels of perlecan, the major extracellular HSPG. The role of perlecan in LDL uptake was further confirmed by using anti-perlecan antibodies, which significantly inhibited LDL uptake by liver cells.

Role of Peroxiredoxin on Liver Cell LDL Uptake:

The Peroxiredoxin family of peroxidases are the principal enzymes involved in regulating the receptor generated H₂O₂ and Peroxiredoxin I is the most abundant enzyme in liver. RNAi mediated knock-down of Peroxiredoxin resulted in a 60% decrease in Peroxiredoxin mRNA. This was associated with a significant 50% decrease in perlecan mRNA without changes in LDLr mRNA levels. This effect seems to be highly specific to Peroxiredoxin as catalase knock down had no significant effect on either LDLr or perlecan mRNA. Liver cells with reduced Peroxiredoxin expression showed significantly diminished LDL uptake (45% decrease, p<0.01). These data further confirm a role for endogenous Peroxiredoxin 1 and H₂O₂ signaling in modulating LDL uptake.

Role of Stat1 in H₂O₂ Mediated Suppression of Perlecan Expression and LDL Uptake:

H₂O₂ is known to target cysteine residues on protein tyrosine phosphatases leading to sustained activation of signaling kinases and transcription factors including Stat1. Stat1 has been shown to be a transcriptional suppressor of perlecan; therefore, the effects of H₂O₂ and Peroxiredoxin on Stat1 activity were determined. Addition of H₂O₂ or Peroxiredoxin knock-down significantly enhanced Stat1 driven luciferase expression. Adenoviral mediated overexpression of Stat1 in liver cells significantly decreased perlecan expression and LDL uptake. These data demonstrate a negative role for Stat1 in liver cell LDL uptake via perlecan expression.

Role of Peroxiredoxin on Circulating Plasma Lipoproteins:

To further understand the role of Peroxiredoxin I in LDL metabolism, the present invention used in vivo RNAi methods to knock down Peroxiredoxin I expression in liver. Intravenous injection of Peroxiredoxin RNAi in mice resulted in a 60% decrease Peroxiredoxin I mRNA. This was associated with a significant 25% reduction in perlecan mRNA. Importantly, within 7 days of Peroxiredoxin RNAi treatment, plasma levels of total cholesterol was significantly increased (14%, p<0.05. Liver apoB mRNA levels were not affected, however, there was a marked increase in plasma apoB protein levels in Peroxiredoxin I KO mice (a 65% increase. This data shows the impact of Peroxiredoxin I mediated regulation of H₂O₂ signaling in LDL metabolism.

### Example 1

This example illustrates the role of Peroxiredoxin in LDL metabolism.

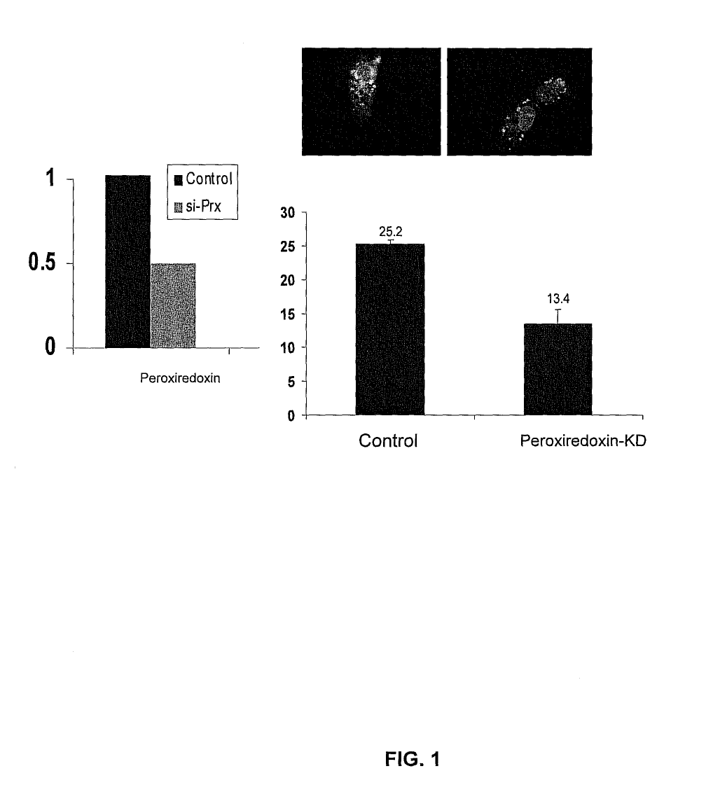

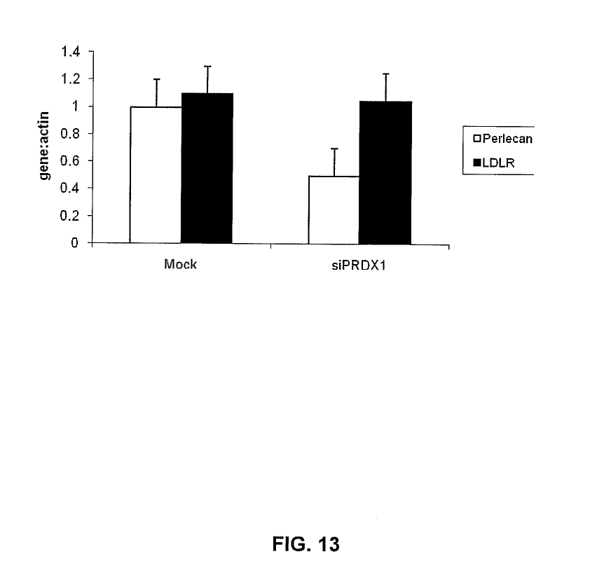

To determine the role of Peroxiredoxin on LDL metabolism in liver cells a siRNA (RNAi) gene silencing approach was used to reduce Peroxiredoxin expression (“Peroxiredoxin knock-down” or “Peroxiredoxin KD”). HepG2 (Human hepatocellular liver carcinoma cell line) were used as a representative of liver cell. Liver cells were incubated with siRNA designed to knock-down Peroxiredoxin 1 or a non-specific siRNA and at the end of a forth eight hours incubation time, Peroxiredoxin levels in the cells were determined by quantitative PCR. In this test, Peroxiredoxin expression was reduced by siRNA (RNAi) gene silencing by 50%. Knocking down the Peroxiredoxin gene resulted in a decreased uptake of labeled LDL by liver cells (determined by fluorescent microscopy and quantitated. These data demonstrate that Peroxiredoxin promotes LDL clearance by liver cells. FIGS. 1 and 13 illustrate the results from Example 1 in which the Peroxiredoxin activity knock-down experiment decreased LDL clearance by liver cells.

### Example 2

This example illustrates the role of Peroxiredoxin on LDL in an in vivo mouse model.

Mice were injected with siRNA for Peroxiredoxin. Three daily consecutive treatments of siRNA were administered in C57BL/6 mice by tail vein injection. Fasted blood samples were collected from each animal at baseline, and at the end of the study.

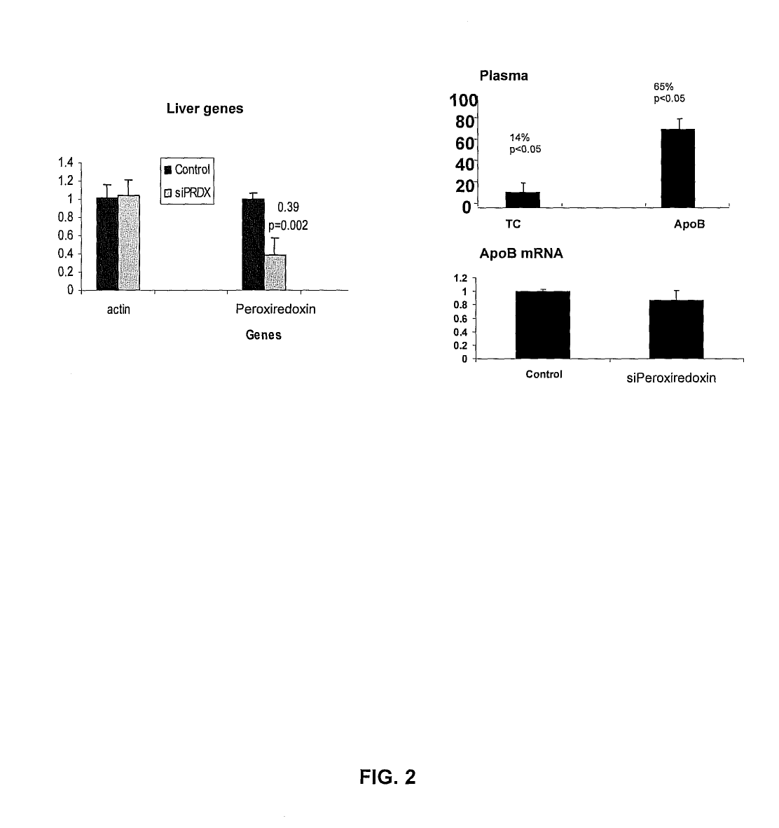

Total cholesterol (“TC”) and apoB levels were measured using an enzymatic assay. Percentage change was evaluated by comparing to both the baseline and the vehicle at the end of the study. In addition, liver tissue was collected at the end of the study. RNA was isolated according to the manufacturer's protocol (Qiagen) and analyzed for changes in gene expression normalized to actin using RT-PCR. The mice later showed reduced levels of Peroxiredoxin activity. This decrease in Peroxiredoxin was associated with an increase in plasma concentrations of total cholesterol and apolipoprotein B (apoB). ApoB mRNA levels in liver cells were not affected suggesting that the liver cell production of apoB was not affected but clearance of apoB lipoproteins from plasma was decreased resulting increased plasma cholesterol. Therefore, Peroxiredoxin has a role in reducing plasma cholesterol and reducing apoB-containing lipoproteins. FIG. 2 illustrates the results from Example 2 in which a Peroxiredoxin activity knock-down experiment in animals increased plasma LDL and apoB concentrations.

### Example 3

This example illustrates the therapeutic use of Peroxiredoxin inducers in animal subjects suffering from hyperlipidemia.

An assay was developed that identified Peroxiredoxin inducers, which activated Peroxiredoxin and demonstrated treatment efficacy for cardiovascular diseases, such as hypercholesterolemia and hypertriglyceredemia.

An in vitro assay was developed to identify compounds that activated Peroxiredoxin (Peroxiredoxin inducers). In a 96-well plate, Peroxiredoxin (0.1 μg) was incubated with 5 μM of an unknown candidate compound and mixed with Hepes buffer containing NADPH, thioredoxin, thioredoxin reductase and hydrogen peroxide. When hydrogen peroxide was reduced by the activated Peroxiredoxin, the quantitative indicator, NADPH was converted to NADP. A decrease in NADPH was monitored by determining the absorption at 360 nm. A decrease in NADPH was therefore, indicative of a Peroxiredoxin inducer.

This example illustrates a sample Peroxiredoxin assay.

Peroxiredoxin-mediated reduction of H₂O₂ was coupled to oxidation of NADPH oxidation. NADPH oxidation was monitored as a decrease in absorbance at 340 nm (A₃₄₀). The control reaction contained 50 mM Hepes-NaOH (pH 7.0), 1 mM EDTA, 2 μM thioredoxin, 150 nM thioredoxin reductase, H₂O₂ (8.8 mM), NADPH (8 mM), and Peroxiredoxin (1 μM). The treatment group in addition contained 500 nM of compound A. The reaction was incubated for 30 seconds to 10 minutes and the change in absorbance was monitored.

Measurement of Cellular Peroxide Levels:

H₂O₂ levels in human aortic smooth muscle cells (Rockland, Me. Clonetics, catalog #CC-2535) were measured using Amplex Red Kit (Molecular Probes). Smooth muscle cells were cultured in Clonetics growth media containing 5% fetal bovine serum. Cells at 70 percent confluence were starved overnight in serum free media and incubated for further 30 min in serum free media alone (control) or medium containing 2 μM compound A (treatment) for 30 minutes. Growth media containing serum was then added and incubated for one hour. The cells were then lysed and H₂O₂ was assayed in lysates using Amplex Red Kit (Molecular Probes).

MCP-1 ELISA:

MCP-1 ELISA is carried out using Quantikine Human MCP-1 kit as described by manufacturer (R&D Systems, Minneapolis, Minn., Catalog #DY279E). Mouse anti-human MCP-1 was used as the capture antibody and HRP-conjugated goat anti-human MCP-1 (Zymed (now part of Invitrogen) catalog #81-1620, Carlsbad, Calif.) was used as the detection antibody. Culture media were incubated with capture antibody (in 96 well) for 2 hours at room temperature. Wells were washed three times with wash buffer (0.05% tween-20 in phosphate buffered saline (PBS) pH 7.4) followed by incubation with detection antibody for 2 hours at room temperature. Color development was read at 450 nm in a Microplate reader.

VCAM-1 ELISA:

Endothelial cell surface VCAM-1 was measured following compound A treatments. The cell layer was washed once with PBS and fixed with methanol. After fixing the cells, VCAM-1 expression was detected with the primary VCAM-1 antibody (goat-anti-Human VCAM-1 antibody—catalog# BBA19, R&D Systems) and secondary anti-goat antibody conjugated to horse radish peroxidase. Colorimetric measurements with made with the Multiskan Ascent plate reader.

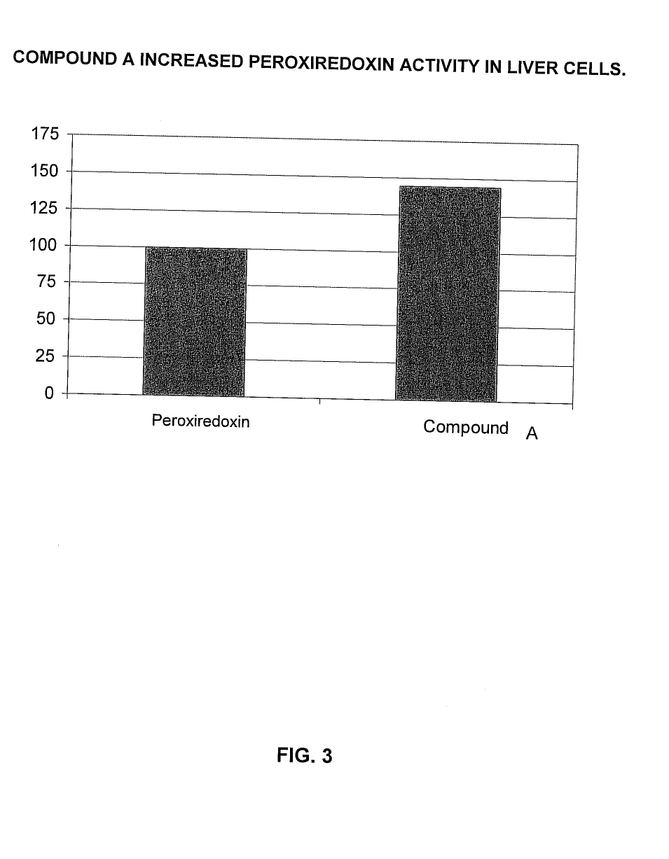

FIG. 3 illustrates the results from Example 3 in which Compound A increased Peroxiredoxin activity in liver cells. The addition of Compound A to liver cells, as shown in FIG. 3, increased Peroxiredoxin activity by 48% over controls without the addition of Compound A.

### Example 4

This example illustrates that a Peroxiredoxin inducer demonstrated a cholesterol and triglyceride lowering effect in animal models of hyperlipidemia.

Compound A, which induced Peroxiredoxin, was tested in two rodent models of hyperlipidemia (LDL-receptor null and apoE null mouse model).

First, 14 week old LDL-receptor null mice were fed with a diet containing 0.8% cholesterol, 0.1% cholic acid and 20% coconut oil and once a day orally dosed with carboxymethyl cellulose suspension alone Compound A at 100 mg/kg (in carboxymethyl cellulose suspension) for 15 days. At the end of the study, plasma levels of cholesterol and triglycerides were determined using an enzymatic method (INFINITY™ Sigma Diagnostics).

Second, 8 week old apolipoprotein E-null mice were fed with normal chow diet and once a day orally dosed with carboxymethyl cellulose suspension alone or Compound A at 100 mg/kg (in carboxymethyl cellulose suspension) for 14 days. At the end of the study, plasma levels of cholesterol and triglycerides were determined.

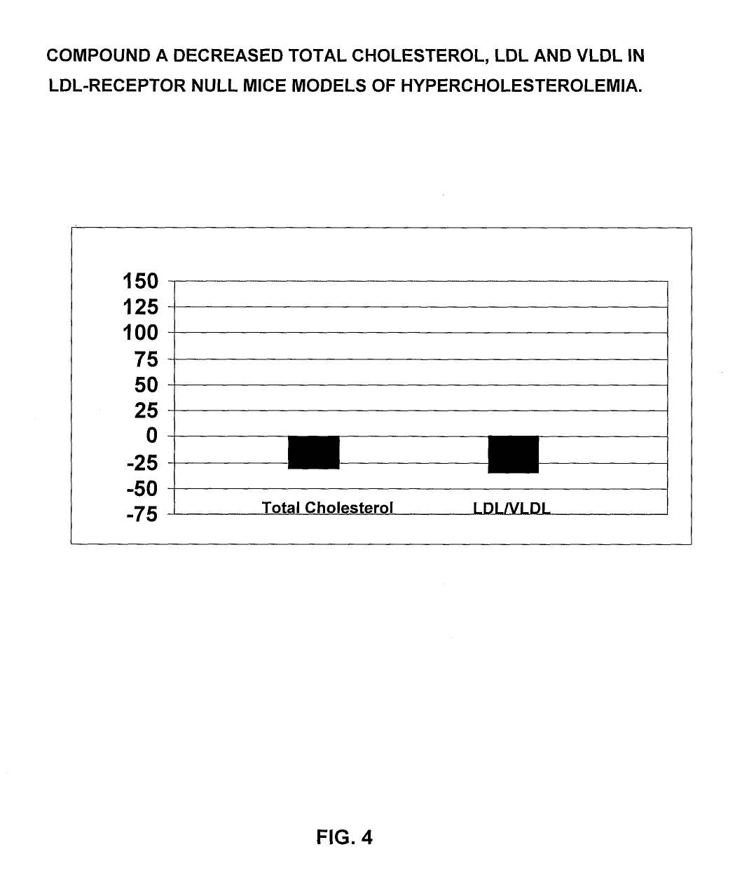

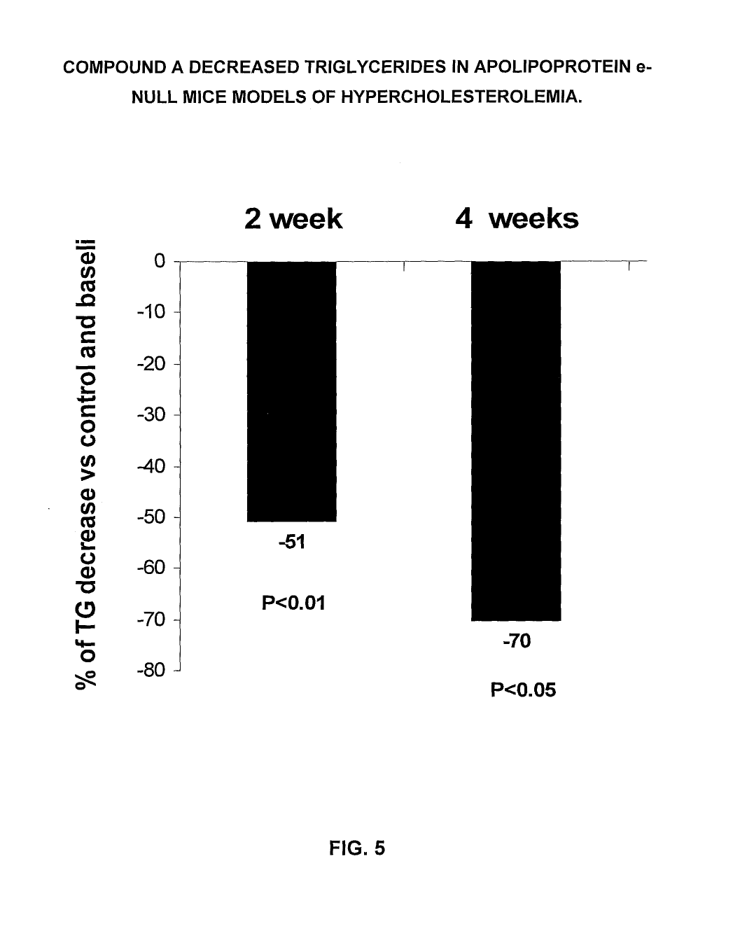

In both models, compound A showed beneficial effects on lipoproteins that are known to cause cardiovascular disease (see FIGS. 4 and 5) compared to vehicle-treated control animals. There was a reduction total cholesterol, LDL and VLDL cholesterol and triglycerides in LDL-receptor null mice treated with compound A. In apoE-null mice treated with compound A, there was a reduction in total triglycerides. Thus, Peroxiredoxin activation demonstrated beneficial effects on hyperlipidemia.

FIG. 4 illustrates the results from Example 4 in which compound A decreased total cholesterol, LDL and triglycerides in LDL-receptor null mice models of hypercholesterolemia. FIG. 5 also illustrates the results from Example 4 in which Compound A decreased triglycerides in apolipoprotein E-null mice models of hypercholesterolemia.

### Example 5

This example illustrates methodology used in Example 1-4.

Liver Cell Cultures:

A hepatoma cell line HepG2 (available from ATCC) was used as an in vitro model of liver cells because they retain many properties of liver cells including expression of various genes involved in lipid metabolism. They were cultured under standard conditions in T-75 flasks prior to the study.

RNA Isolation and Real Time PCR:

Liver and aorta samples from vehicle treated and compound A treated mice were removed, flash frozen in liquid nitrogen and subsequently used for RNA isolation. Tissues were lysed in 600 uL lysis buffer (Qiagen) and placed in the TissueLyser (Qiagen) for 3 minutes. Samples were then processed using the RNeasy mini kit (liver) or the RNeasy fibrous tissue mini kit (aorta) (Qiagen, Valemcia, Calif.). RNA was then verified and quantified using the Agilent RNA 600 Nano Assay Labchip® system, and real time PCR was performed to quantitate the gene expression of Peroxiredoxin using validated primer sets from SuperArray.

Determination of LDL Uptake:

6-well tissue culture plates containing sterile glass cover slips were seeded with 75000 cells per well in a total of 1 ml per well in HepG2 growth media. Cells were allowed to adhere and grow for 24 hours. Second day cells were pretreated with compound A in growth media. Seeding media was removed and 1 ml of growth media+either compound A or DMSO control was added to wells. The pre-treatment was for 24 hours. The third day treatments were continued in basal media+10% delipidated calf serum (DLCS). The pretreatment media was removed and the wells were washed 3 times with basal media. After washes, 1 ml of basal media+10% (DLCS)+either compound A or DMSO control was added to wells and treatments continued for 24 hours. On the fourth day, the media was not changed and Dil LDL and DAPI staining were completed. 2 microliters of Dil LDL from molecular probes was added to each well. 0.25 microliters of DAPI was added to each well and the cells were allowed to incubate another 5 hours for the uptake.

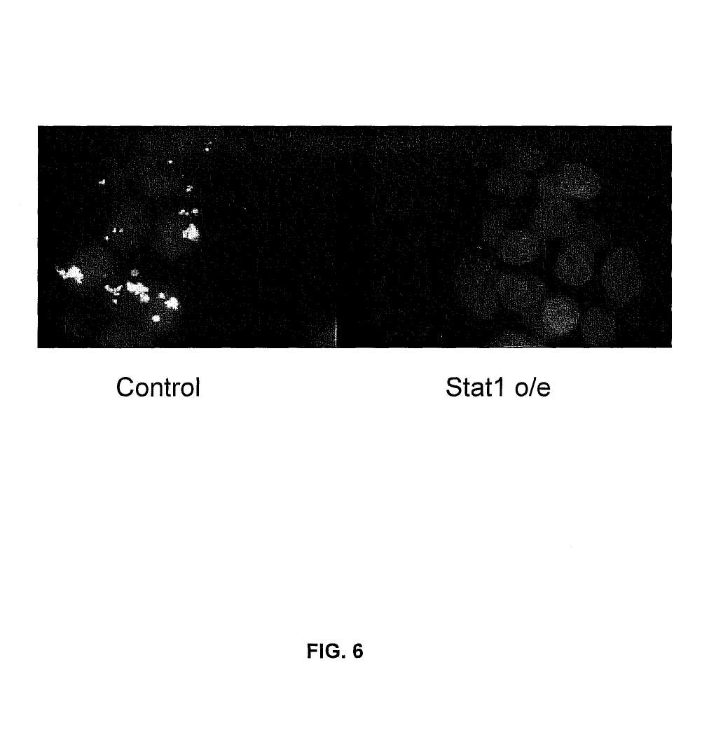

After the uptake was completed, the wells were washed gently 3 times with PBS (2 mL/well/wash) and after last wash cells were fixed with 500 microliters of 4% formaldehyde in PBS for 20 minutes. The wells were again washed 5 times with water in excess. Cover slips were then mounted to slides using Zymed clearmount. Slides were stored at −20° C. The slides were brought to room temperature and images were captured using a digital camera attached to a microscope. FIGS. 1 and 6 are representative of these images and methodology.

### Example 6

This example illustrates the role of H₂O₂ and Perlecan in LDL uptake by liver cells.

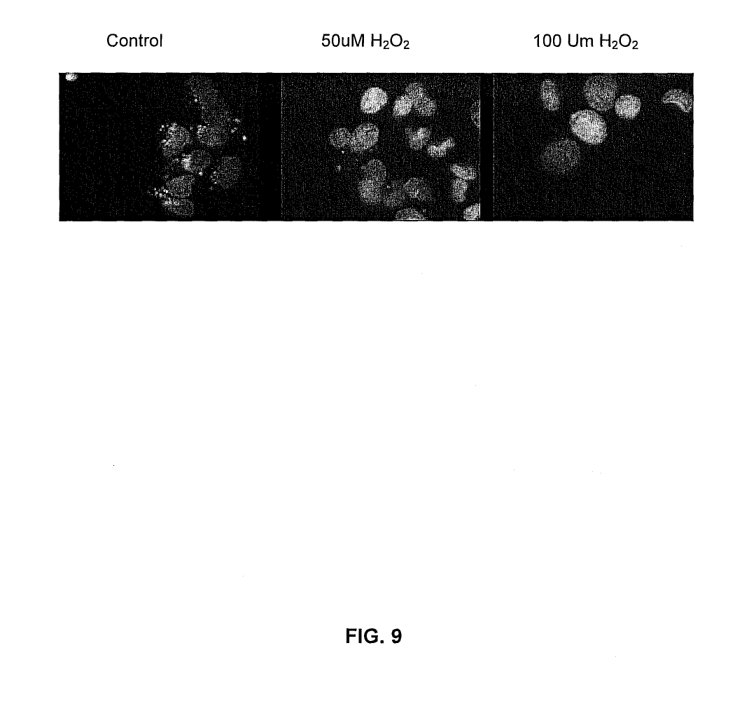

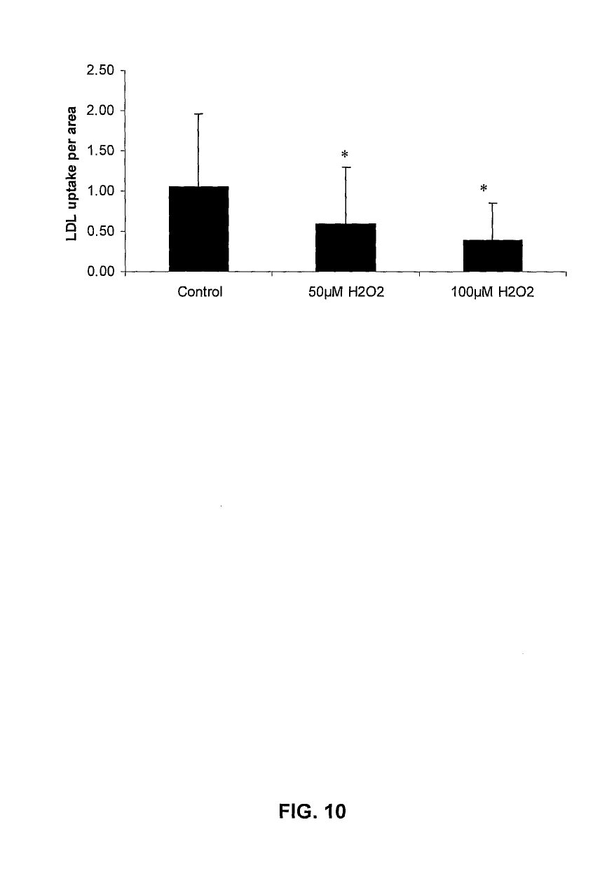

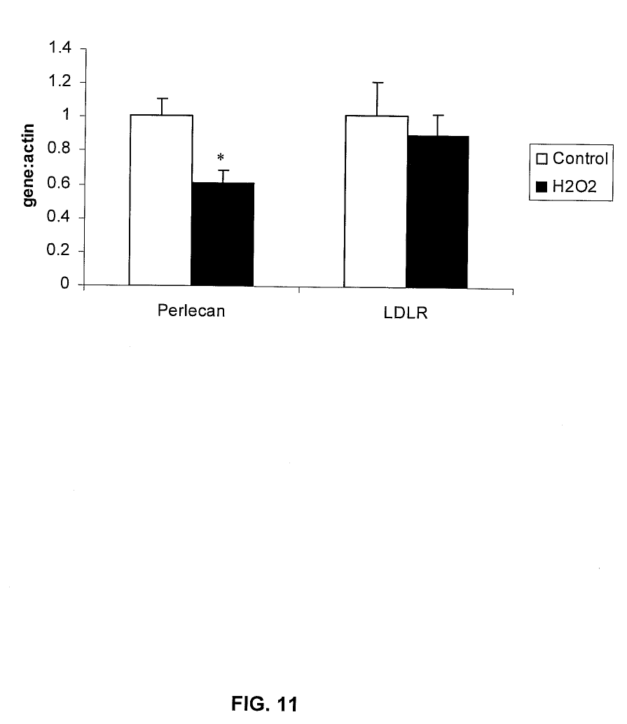

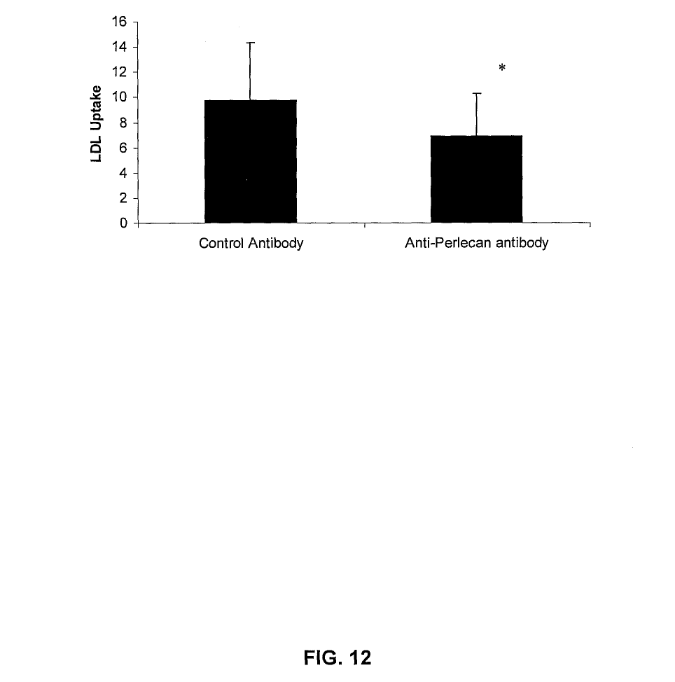

HepG2 cells were treated with either vehicle, 50 μM H₂O₂, or 100 μM H₂O₂, and LDL uptake was measured as described above. HepG2 cells were treated with either vehicle or 100 μM H₂O₂. RNA was isolated and gene analysis was performed by RT-PCR. HepG2 cells were pretreated with either Control IgG or anti-perlecan antibodies prior to LDL uptake. Bars show the mean±SD. Asterisks indicate statistical difference from the Control group with significance values of p<0.05. FIGS. 9-12 illustrate the role of H₂O₂ and Perlecan in LDL uptake by liver cells.

### Example 7

This example illustrates that H₂O₂ and Peroxiredoxin I regulate Stat1 activity.

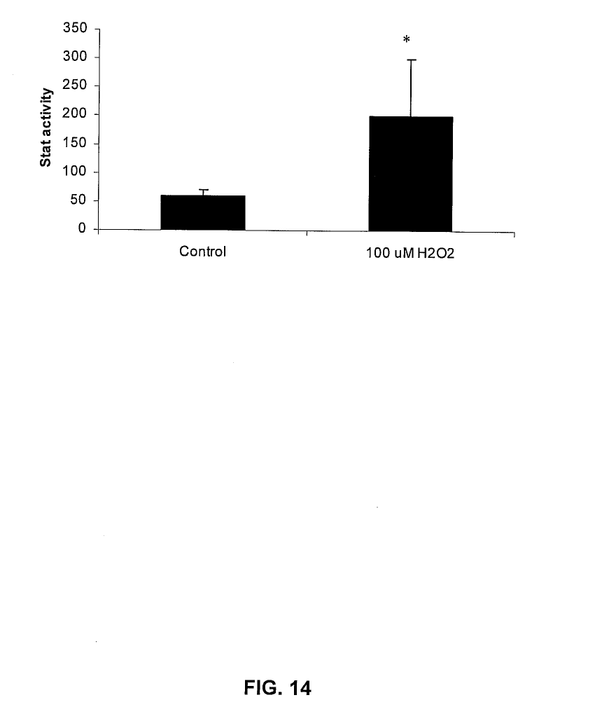

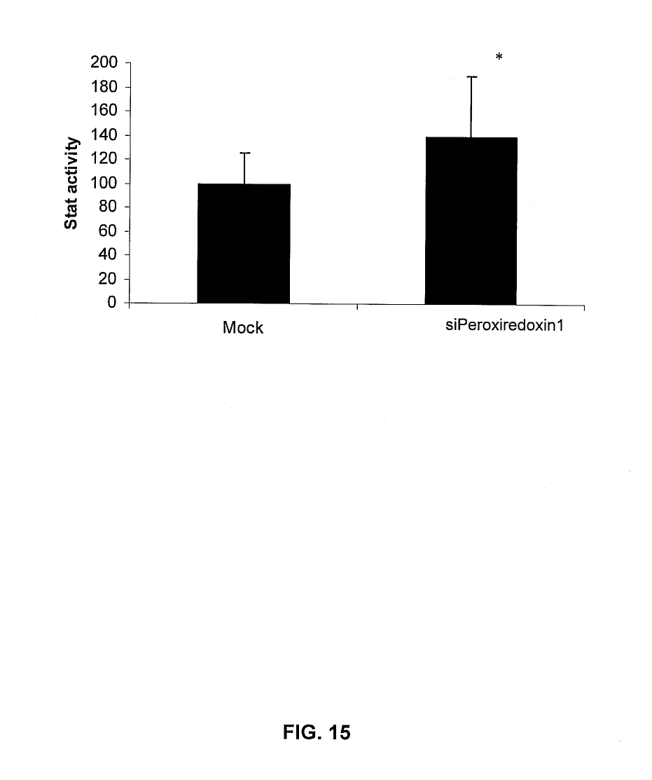

HepG2 were transiently transfected with a Stat1-luciferase reporter construct and subject to treatment with either H₂O₂ or siPeroxiredoxin 1. Bars show the mean±SD. Asterisks indicate statistical difference from the Mock group with significance values of p<0.05. FIGS. 14 and 15 illustrate that H₂O₂ and Peroxiredoxin I regulate Stat1 activity.

### Example 8

This example illustrates the role of Stat1 on LDL uptake.

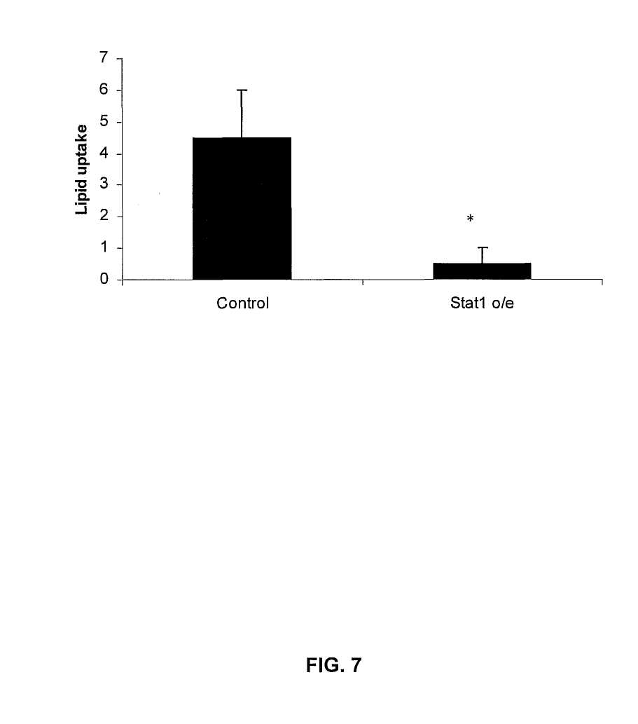

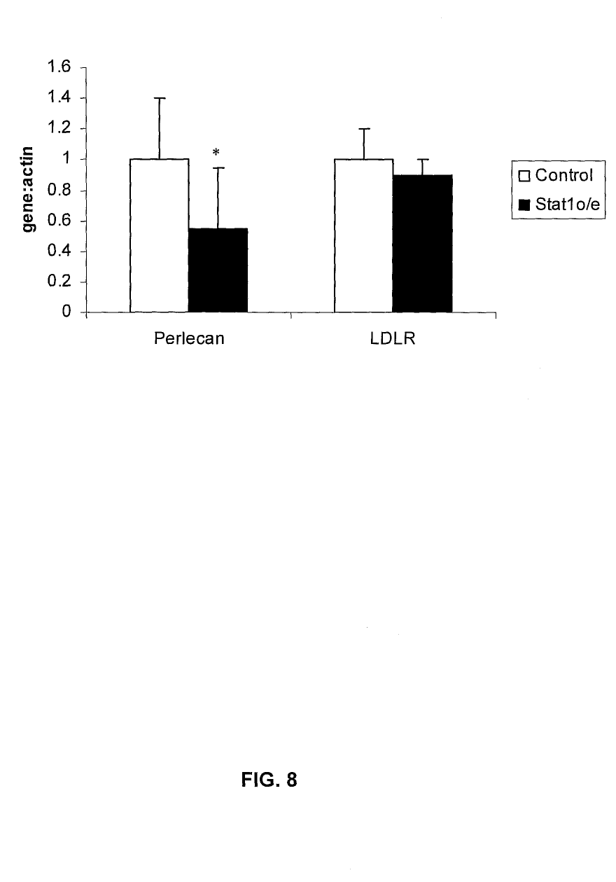

HepG2 cells were subject to adenoviral mediated overexpression of Stat1. LDL uptake was measured as described above. RNA was isolated and gene analysis was performed by RT-PCR. Bars show the mean±SD. Asterisks indicate statistical difference from the Control group with significance values of p<0.05. FIGS. 6 and 7 illustrate the results of Example 8.

### Example 9

This example illustrates that Peroxiredoxin-activating compounds decrease hydrogen peroxide levels in cells.

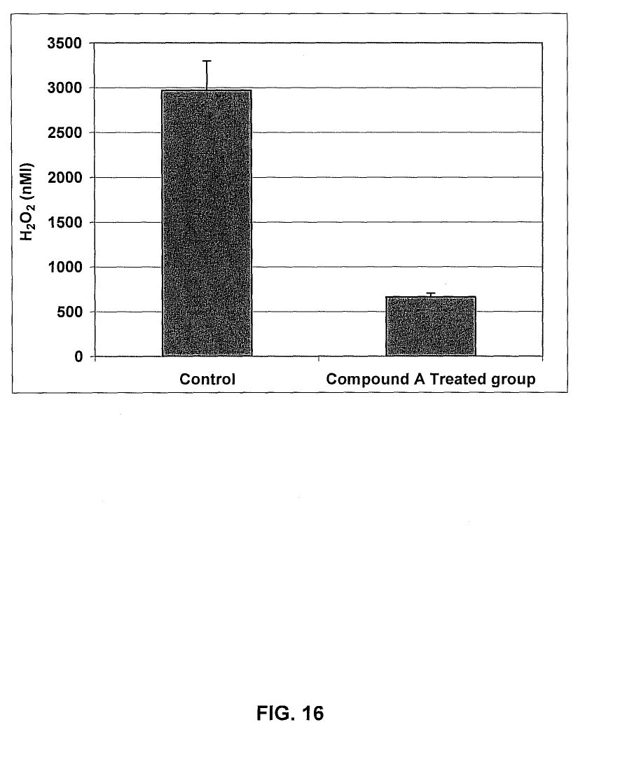

To establish that compound mediated activation of Peroxiredoxin will result in decreased H₂O₂, H₂O₂ levels were measured in untreated (control) and compound A treated smooth muscle cells (See FIG. 16). Human Aortic Smooth Muscle cells at 70 percent confluence were starved overnight in basal media and incubated for further 30 min in basal medium alone (control) or medium containing 3 uM compound (treatment) for 30 minutes. Growth media was then added to the control sample and growth media plus compound was added to the treatment group and incubated for one hour. The cells were then lysed and H₂O₂ was assayed in lysates using Amplex Red Kit (Molecular Probes).

H₂O₂ levels were decreased by approximately 77% in compound treated group compared to untreated control group suggesting that the Peroxiredoxin-activating compound A induced activation of Peroxiredoxin had a functional effect on cellular peroxide levels.

### Example 10

This example illustrates that Peroxiredoxin-activating compounds inhibit inflammatory cytokine expression in endothelial cells.

Inflammation has been shown to play a role in the pathogenesis of cardiovascular diseases. TNF-alpha (TNFα) (R&D Systems, Minneapolis, Minn., Catalogue No. 210-TA-010) is a pro-inflammatory cytokine implicated in the development of cardiovascular disease. Inflammatory markers produced in response to TNFα include monocyte chemoattractant protein—MCP-1, and vascular cell adhesion molecule—VCAM-1. Endothelial cells are known to produce these markers in response TNFα. To determine whether compound mediating the activation of Peroxiredoxin will result in decreased inflammation, MCP-1 and VCAM-1 levels were measured in TNFα-induced endothelial cells. Cells were stimulated with 0.5 ng/ml of TNFα with or without compound A in basal media supplemented with 1% FBS. Each treatment condition was done in triplicate. After overnight treatment (about 15-18 hours), the cell culture media was removed and used for MCP-1 ELISA as described in the manufacturer's protocol. The cell layer was washed once with PBS and fixed with methanol. After fixing the cells, VCAM-1 expression was detected with the primary and secondary antibodies. Colorimetric measurements with made with the Multiskan Ascent plate reader.

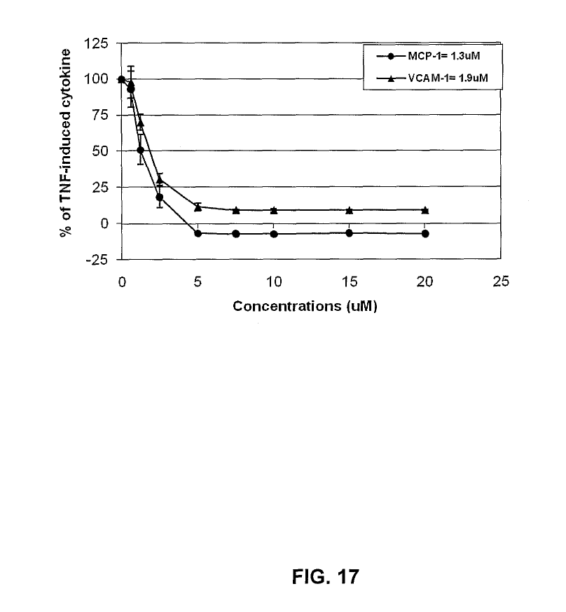

MCP-1 and VCAM-1 levels were decreased in the compound A treated group compared to the untreated control group (0 μM). At 2.5 μM concentration, the Peroxiredoxin-activating compound A inhibited MCP-1 and VCAM-1 levels by 75%, suggesting that such compounds induced activation of Peroxiredoxin had a functional effect on inflammatory gene expression. FIG. 17 illustrates the results from Example 10.

### Example 11

This example illustrates that Peroxiredoxin-activating compounds inhibit inflammatory cytokine expression in animal models of acute inflammation.

The anti-inflammatory effect of Peroxiredoxin-inducing compound A on TNF-α elevation in a rat model of acute inflammation was demonstrated. The compound A was orally administrated (60 mg/kg) to female Sprague Dawley rats followed by intraperitoneal administration lipopolysaccharide (LPS−1 mg/kg). Blood samples were collected 1 hour after LPS administration. TNF-α level in the serum was analyzed by using ELISA.

Compared to untreated animals (TNFα levels undetectable), LPS induced elevation of TNFα in serum (TNFα levels 2353.7 pg/ml). The Peroxiredoxin-activating compound A, at 60 mg/kg, completely inhibited TNFα elevation that was induced by LPS (undetectable in Peroxiredoxin-activating compound-treated rats). This example shows that compound A-induced activation of Peroxiredoxin inhibits inflammatory gene expression in vivo.

In view of the above, it will be seen that the several advantages of the invention are achieved and other advantageous results obtained.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

As various changes could be made in the above methods and compositions by those of ordinary skill in the art without departing from the scope of the invention, it is intended that all matter contained in the above description and shown in the accompanying drawings shall be interpreted as illustrative and not in a limiting sense. In addition it should be understood that aspects of the various embodiments may be interchanged both in whole or in part.

1. A method of identifying compounds capable of upregulating Peroxiredoxin activity, the method comprising:
providing a sample of cells that express Peroxiredoxin;
providing a sample of a candidate compound;
contacting the cell sample and the candidate compound; and
measuring Peroxiredoxin activity within the cell sample after the contacting step.

2. The method according to claim 1, wherein the step of measuring Peroxiredoxin activity comprises measuring a quantitative indicator of Peroxiredoxin activity.

3. The method according to claim 1, wherein the cell sample is contacted with the candidate sample in the presence of a high-throughput Peroxiredoxin activity assay based on NADPH concentration.

4. The method according to claim 1, wherein the cells that express Peroxiredoxin are human liver cells.

5. The method according to claim 1, wherein the contacting step is in vitro.

6. The method according to claim 2, wherein the quantitative indicator of Peroxiredoxin activity is NADPH concentration.

7. The method according to claim 2, wherein the quantitative indicator of Peroxiredoxin activity optionally comprises the measurement of posttranslational Peroxiredoxin activity.

8. A method of identifying compounds that lower serum LDL and/or VLDL levels in a subject, the method comprising:
providing a sample of cells that express Peroxiredoxin;
providing a sample of a candidate compound;
contacting the cell sample and the candidate compound;
measuring Peroxiredoxin activity within the cell sample after the contacting step; and
selecting those candidate compounds that increases Peroxiredoxin activity as compounds that lower serum LDL and/or VLDL levels in the subject.

9. A method of lowering serum LDL and/or VLDL levels in a subject comprising administering to the subject a Peroxiredoxin inducer.

10. A method of lowering serum triglyceride levels in a subject comprising administering to the subject a Peroxiredoxin inducer.

11. A method of reducing total and LDL-cholesterol in a cell of a subject, comprising increasing the amount and/or activity of Peroxiredoxin within the cell, wherein total and LDL-cholesterol levels are reduced.

12. A method of treating or preventing hypercholesterolemia and/or hypertriglyceredemia, comprising administering to a subject an effective amount of a compound that causes an increase in the amount and/or activity of Peroxiredoxin.

13. A method for diagnosing a dyslipidemia condition in a subject by measuring the activity of Peroxiredoxin and correlating the activity with a known dyslipidemia condition.

14. A method of treating a disorder associated with an increase in inflammatory cytokines, which method comprises increasing the activity of Peroxiredoxin.

15. The method according to claim 14, wherein the inflammatory cytokines is TNFα, MCP-1 or VCAM-1.

16. A method of treating a disorder associated with an increase in inflammatory cytokines, which method comprises upregulation of the Peroxiredoxin gene.

17. A method of treating a disorder associated with an increase in inflammatory cytokines, wherein the inflammatory cytokines is TNFα, or VCAM-1. In another aspect the present invention provides a method of treating a disorder associated with an increase in inflammatory cytokines, wherein the disorder is an inflammatory disorder.

18. A method of treating a disorder associated with an increase in inflammatory cytokines, wherein the disorder is a cardiovascular disorder.

19. A method of treating a disorder associated with an increase in inflammatory cytokines, wherein the disorder is a metabolic disorder.

20. A method of treating a disorder associated with an increase in inflammatory cytokines, wherein the disorder is diabetic nephropathy.

21. A method for the screening of compounds that modulate the activity of Peroxiredoxin.

22. A method of identifying whether or not a compound is capable of increasing the activity of Peroxiredoxin.

23. A method for the screening and identification of compounds that provoke the activity of Peroxiredoxin, comprising (a) incubating an effective amount of the compound of interest together with Peroxiredoxin, under conditions sufficient to allow the components to interact; and (b) screening and identifying the compound by measuring the oxidation of NADPH.

24. A method for the screening and identification of compounds that provoke the activity of Peroxiredoxin, comprises (a) incubating an effective amount of the compound of interest together with Peroxiredoxin, NADPH, EDTA, thioredoxin, thioredoxin reductase, and Hepes-NaOH, under conditions sufficient to allow the components to interact; and (b) screening for activation of Peroxiredoxin and identifying the compound by measuring the oxidation of NADPH.

25. A method for the treatment of inflammatory-induced disease and cardiovascular disorders comprising administering to a subject in need thereof a therapeutic effective amount of a compound that increases the activity of Peroxiredoxin or a pharmaceutically acceptable salt thereof.

26. The method according to claim 25, wherein the inflammatory-induced disease is selected from the group comprising of arthritis, asthma, atherosclerosis, irritable bowel syndrome, Crohn's disease, type 2 diabetes, psoriasis, diabetic nephropathy, retinopathy, and glomeluar nephritis.

27. A method for treating a disease state which is alleviable by the treatment with a compound that affects the activity of Peroxiredoxin, comprising administering to a subject in need thereof a therapeutic effective amount of a compound that increases the activity of Peroxiredoxin or a pharmaceutically acceptable salt thereof.

28. A method for the treatment of inflammatory and cardiovascular disorders comprising providing to a subject in need of treatment an effective amount of a compound that increases the activity of Peroxiredoxin.

29. Use of a compound that increases the activity of Peroxiredoxin for the manufacture of a medicament for the treatment of inflammatory and cardiovascular disorders.

30. A method of treatment comprising administering a compositing containing a purified amount of a compound that increases the activity of Peroxiredoxin.
